(19) United States
(12) Patent Application Publication
(10) Pub. No.: US 2006/0148046 A1
(43) Pub. Date: Jul. 6, 2006

(54) Enzymatic process for the preparation of an intermediate compound and use thereof for the synthesis of tamsulosin hydrochloride

(75) Inventors: Javier Villasante Prieto, Madrid (ES); Francisco Palomo Nicolau, Alcala De Henares (ES)

Correspondence Address: WOLF BLOCK SCHORR AND SOLIS-COHEN LLP, 250 PARK AVENUE, NEW YORK, NY, 10177, US

(73) Assignee: Quimica Sintetica, S.A.

(21) Appl. No.: 11/079,863

(22) Filed: Mar. 14, 2005

(30) Foreign Application Priority Data
Dec. 31, 2004 (ES) P-200403165

(51) Int. Cl. C12P 13/02

(52) U.S. Cl. 435/129

(57) ABSTRACT

The present invention relates to a process of enantioselective enzymatic acylation for the preparation of (R)-2-halo-N-[2-(4-methoxyphenyl)-1-methyl-ethyl] acetamide compounds, of formula III, in which X═Cl, Br, I.
These compounds (III) are useful as intermediates in the synthesis of tamsulosin hydrochloride.

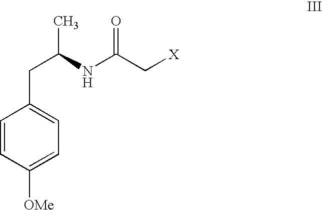

### FIELD OF THE INVENTION

This invention relates to the preparation of intermediate compounds, (R)-2-halo-N-[2-(4-methoxyphenyl)-1-methylethyl]acetamide, by means of an enantioselective enzymatic acylation process. These intermediate compounds are useful in the synthesis of tamsulosin hydrochloride. Preferably, the invention relates to the preparation of the compound (R)-2-chloro-N-[2-(4-methoxyphenyl)-1-methylethyl]acetamide.

### PRIOR ART

Tamsulosin hydrochloride is the international nonproprietary name of (R)-5-[2-[[2-(2-ethoxyphenoxy)ethyl]amino]propyl]-2-methoxybenzenesulphonamide hydrochloride of Formula (I), with application in medicine and used in the treatment of benign prostatic hypertrophy.

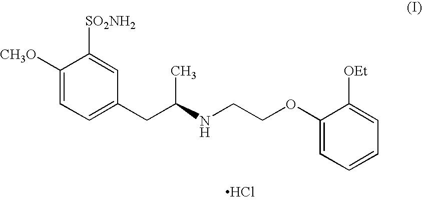

Racemic tamsulosin hydrochloride was disclosed for the first time in European patent EP 34.432-B1. The equivalent North American patents U.S. Pat. No. 4,731,478 and U.S. Pat. No. 4,761,500 specifically disclose the R-enantiomer.

The preparation of tamsulosin disclosed in these patents U.S. Pat. No. 4,731,478 and U.S. Pat. No. 4,761,500, in Spanish patent ES 2000382, in European patent EP 257787-B1 and in PCT application no. WO 03/035608 A1 is based on the use of the intermediate compound R(−)-5-[(2-amino-2-methyl)ethyl]-2-methoxy benzenesulphonamide (VI), which by reaction with an o-ethoxyphenoxy derivative yields tamsulosin.

In North American patents U.S. Pat. No. 4,731,478 and U.S. Pat. No. 4,761,500 and in Spanish patent ES 2000382, this intermediate compound VI is prepared from (R)-4-methoxyamphetamine, while in patent EP 257787-B1 and in PCT application no. WO 03/035608 A1 it is prepared using a chiral amine VII:

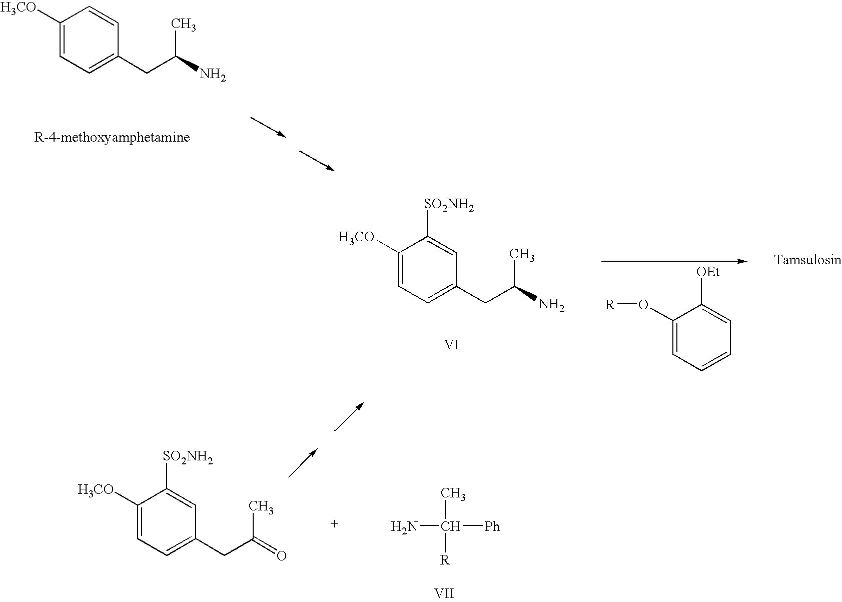

All these processes require the obtention of the enantiomerically pure intermediate compound VI, from which tamsulosin hydrochloride is obtained. The prior methods used for the preparation of this intermediate VI require many reaction steps, which hinder good industrial implantation and result in low yields. Furthermore, the starting compound (R)-4-methoxyamphetamine and the chiral amines used in the preparation of the intermediate VI are very costly reagents.

Japanese patents JP 02306958 and JP 02295967 disclose a process for the preparation of tamsulosin hydrochloride consisting on the preparation of a chiral intermediate VIII from (R)-4-methoxyamphetamine by means of acylation with bromo acetic acid/t-butylic acid chloride/Et₃N to obtain the (R)-bromo-N-[2-(4-methoxyphenyl)-1-methylethyl]-acetamide VIII. This compound VIII is reacted with chlorosulphonic acid and is subsequently treated with NH₃ to yield the sulphonamide IX derivative, which is reacted with 2-ethoxyphenol to yield an amide X which is reduced with lithium aluminium hydride; subsequent treatment with HCl yields tamsulosin HCl:

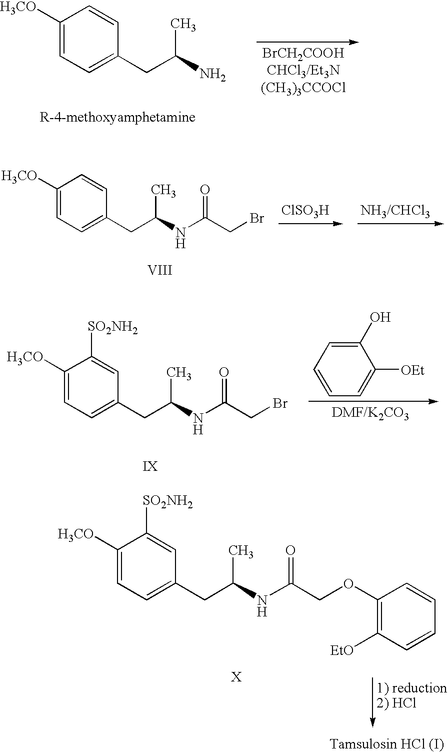

These Japanese patents again use (R)-4-methoxyamphetamine—a highly expensive reagent—as starting product to prepare an enantiomerically pure intermediate compound of formula VIII.

The preparation of (R)-4-methoxyamphetamine is not described in these Japanese patents. The literature, however, contains different processes for its preparation that include several synthesis steps and require the use of expensive starting materials or reagents. Japanese patent JP 09286763-A2, for example, describes the preparation of (R)-4-methoxyamphetamine by optical resolution of the racemate using N-(p-toluenesulphonyl)-L-proline to yield a diastereoisomeric salt which is purified by recrystallisation, followed by decomposition thereof in a basic aqueous solution. In Japanese patent JP 62240651-A2,(R)-4-methoxyamphetamine is prepared by protecting the hydrochloride salt of the ethyl ester of L-tirosine with benzyloxycarbonyl chloride to yield a compound which is treated with methyl iodide and further reduced with NaBH4 to give a compound which is tosylated, then treated with sodium iodide, subjected to reflux with powdered zinc in 1,2-dimethoxyethane and finally deprotected by catalytic hydrogenation. In the publication Campos, F.; Bosch, M. P.; Guerrero, A. Tetrahedron: Asymmetry 2000, 11, 2705-2717, the R-4-methoxyamphetamine is prepared by enantioselective acylation of racemic 4-methoxyamphetamine catalysed by the enzyme Candida antarctica lipase B (CAL-B) with ethyl acetate, separation of the R-acylated compound and subsequent hydrolysis.

New processes are therefore required for preparing optically active compounds useful as intermediates for the manufacture of enantiomerically pure active substances, such as tamsulosin, in a simpler, more efficient and cheaper way.

### OBJECT OF THE INVENTION

This invention relates to a process of enantioselective enzymatic acylation for the preparation of (R)-2-halo-N-[2-(4-methoxy phenyl)-1-methylethyl] acetamide compounds of formula III, wherein X═Cl, Br, I:

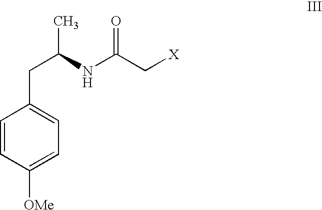

These compounds (III) are useful as intermediates in the synthesis of tamsulosin hydrochloride.

The process of preparation of the optically active compound III of the present invention involves a single reaction step starting from racemic 4-methoxyamphetamine (a cheaper reagent), prevents at least seven-fold consumption of the (R)-4-methoxyamphetamine and the use of expensive reagents for the resolution of the 4-methoxyamphetamine racemate, while it also simplifies the process in relation to the processes described in the state of the art.

### DETAILED DESCRIPTION OF THE INVENTION

The process object of the present invention comprises the enantioselective acylation of racemic 4-methoxyamphetamine with an acylating agent in the presence of an enzyme as catalyst in order to provide a highly stereoselective compound of formula III. Moreover, the compound III thus obtained is used directly, without further treatments, to prepare tamsulosin hydrochloride.

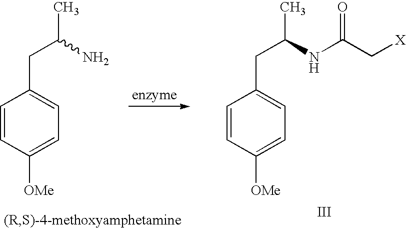

The process is carried out by dissolving the substrate in a suitable solvent and by adding the enzyme and the acylating agent.

Under the action of the enzyme one of the substrate enantiomers is acylated selectively, while the other enantiomer remains largely unacylated.

When the enzymatic process reaches the desired conversion, normally close to 50% in order to obtain the maximum yield of optically enriched acylated product, the reaction is halted, for example by filtering the enzyme off, and the resulting compounds are separated.

The enzyme used in the process object of the present invention is a Lipase, preferably fungi Lipases: Rhizomucor miehei lipase, native freeze-dried (SP 524) and immobilised by adsorption in anionic resin (Duolite A 568) (IM 20), native freeze-dried lipase Thermomyces Lanuginosus (SP 523); yeast lipases: Candida antarctica lipase B, native freeze-dried (SP525) or adsorbed on Lewait E (Novozym 435), native freeze-dried lipase A of Candida antarctica (SP526), native freeze-dried lipase Candida rugosa (CRL); bacterial lipases: native freeze-dried lipase Pseudomonas fluorescens (AK); mammal lipases: native freeze-dried lipase porcine pancreatic (PPL), more preferably the immobilised enzymes Novozym® 435, Lipozyme® TL IM and Lipozyme® RM IM.

The enzymes used are commercial products, available for example from Novozymes France S.A.: Novozym® 435 is a Candida antarctica lipase immobilised in a macroporous acrylic resin; Lipozyme® TL IM is a Thermomyces lanuginosus lipase immobilised on granulated silica, and Lipozyme® RM IM is a Rhizomucor miehei lipase immobilised in a macroporous resin.

The weight/weight ratio between enzyme and substrate can be between 5-20% (w/w), preferably between 10-16% (w/w), more preferably 10% (w/w). The enzyme can be reused, practically retaining its initial activity.

Haloalkyl esters are used as acylating agent, preferably ethyl haloacetate, more preferably ethyl chloroacetate.

An organic inert solvent that maintains the enzyme activity can be used as reaction medium. Examples of organic solvents that can be used in the present invention are organic solvents of the ether type, such as diethyl ether, diisopropyl ether, t-butyl methyl ether, dibutyl ether and tetrahydrofuran; hydrocarbide solvents, such as hexane, heptane, toluene, xylene; ketone-type solvents such as acetone, methyl ethyl ketone and methyl isobutyl ketone. Among these solvents, the ether-type solvents are preferably used, more preferably t-butyl methyl ether.

The reaction temperature can range between 0°-70° C. depending on the activity of the enzyme used. Preferably the reaction temperature will range between 0-40° C., more preferably between 0-5° C. and 15-30° C. The reaction time ranges between 2 h and several days.

The optical purity of the compounds III obtained through the process of the present invention can be determined by HPLC by using chiral columns (column: Chiracel OB-H; eluent: hexane and isopropanol mixture; flow rate: 1.0 ml/min.; detection: 225 nm).

Compounds III, (R)-2-halo-N-[2-(4-methoxy phenyl)-1-methylethyl]acetamide, are used in the preparation of tamsulosin hydrochloride according to the procedure of the following synthesis diagram:

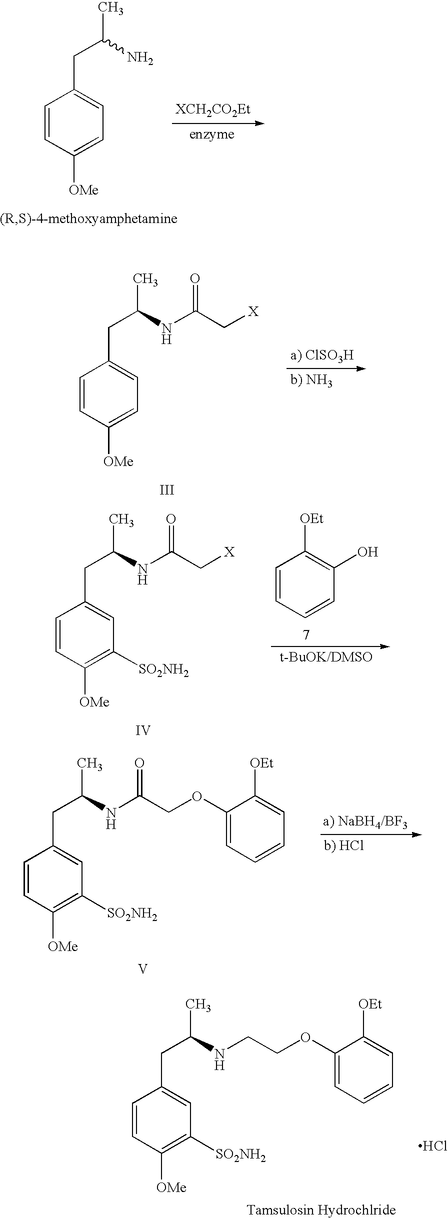

Compound III (R)-2-halo-N-[2-(4-methoxyphenyl)-1-methylethyl] acetamide obtained according to the process of the present invention is treated with chlorosulphonic acid in methylene chloride at a temperature between 15-25° C., followed by treatment with ammonia in acetonitrile at a temperature of (−10)-0° C. to yield (R)-N-[2-(3-aminosulphonyl-4-methoxyphenyl)-1-methylethyl]-2-haloacetamide (IV).

Compound (IV) is reacted with 2-ethoxyphenol in the presence of a strong base, preferably potassium t-butoxide, in an organic solvent, preferably dimethylsulphoxide, at a temperature between 25-35° C. to give rise to compound (V).

Compound (V) is reduced, for example, with NaBH₄/BF₃, in tetrahydrofuran at reflux (approx. 55° C.). Once the reduction reaction has been completed, the obtained compound is treated with hydrochloric acid to yield tamsulosin hydrochloride.

These examples are given solely by way of illustration and do not limit the scope of the invention.

### EXAMPLE 1

### Preparation of (R)-2-chloro-N-[2-(4-methoxyphenyl)-1-methylethyl]acetamide with Novozym 435 not used Previously (Enzyme/Substrate Ratio=16% w/w)

1.60 g of Novozym 435, 100 ml of t-butyl methyl ether (TBME) were placed in a 500 ml flask under N₂ atmosphere and cooled to T=0/5° C. A solution of 32.40 ml (37.13 g; 0.303 mol; 5 eq). of ethyl chloroacetate in 100 ml of TBME was then added, maintaining T =0/5° C. A solution of 10.00 g (0.0605 mol; 1 eq) of (R,S)-4-methoxyamphetamine in 100 ml of TBME was then added. The reaction mixture was maintained under stirring at T=0/5° C. for 2.5 h, following which it was filtered and the retained solid, i.e. a mixture of Novozym 435 and 4-methoxy-α-methylbenzene ethanammonium chloroacetate, was washed with 100 ml of TBME. The filtered solution was washed with H₂SO₄ 0.5 M (2×100 ml) and dried over anhydrous MgSO₄. The dry organic phase was concentrated at reduced pressure and two entrainments were carried out on the resulting concentrate at reduced pressure with 100 ml of heptane at each entrainment, thereby providing 6.30 g of (R)-2-chloro-N-[2-(4-methoxyphenyl)-1-methylethyl]acetamide as a whitish solid.

Yield: 43.1% (R/S)=88.5/11.5

### EXAMPLE 2

### Preparation of (R)-2-chloro-N-[2-(4-methoxyphenyl)-1-methylethyl]acetamide with Novozym 435 not Previously used (Enzyme/Substrate Ratio =10% w/w)

1.00 g of Novozym 435, 100 ml of TBME were placed in a 500 ml flask under N₂ atmosphere and cooled to T=0/5° C. A solution of 32.40 ml (37.13 g; 0.303 mol; 5 eq) of ethyl chloroacetate in 100 ml of TBME was then added, maintaining T=0/5° C. A solution of 10.00 g (0.0605 mol; 1 eq) of (R,S)-4-methoxyamphetamine in 100 ml of TBME was then added. The reaction mixture was maintained under stirring at T=0/5° C. for 3 h, following which it was filtered and the retained solid, i.e. a mixture of Novozym 435 and 4-methoxy-α-methylbenzene ethanammonium chloroacetate, was washed with 100 ml of TBME. The filtered solution was washed with H₂SO₄ 0.5 M (2×100 ml) and dried over anhydrous MgSO₄. The dry organic phase was concentrated at reduced pressure and two entrainments were carried out on the resulting concentrate at reduced pressure with 100 ml of heptane at each entrainment, thereby providing 4.09 g of (R)-2-chloro-N-[2-(4-methoxyphenyl)-1-methylethyl]acetamide as a whitish solid.

Yield: 28% (R/S)=94.7/5.3

### EXAMPLE 3

### Preparation of (R)-2-chloro-N-[2-(4-methoxyphenyl)-1-methylethyl]acetamide with Novozym 435 not used Previously at T=20-25° C. (Enzyme/Substrate Ratio=10% w/w)

0.50 g of Novozym 435 and 50 ml of TBME were placed in a 250 ml flask under N₂ atmosphere. A solution of 16.20 ml (18.57 g; 0.1515 mol; 5 eq) of ethyl chloroacetate in 50 ml of TBME was then added, followed by a solution of 5.00 g (0.0303 mol; 1 eq) of (R,S)-4-methoxyamphetamine in 50 ml of TBME. The reaction mixture was stirred at T=20-25° C. for 21 h, following which it was filtered and the retained solid, i.e. a mixture of Novozym 435 and 4-methoxy-α-methylbenzene ethanammonium chloroacetate, was washed with 50 ml of TBME. The filtered solution was washed with H₂SO₄ 0.5 M (2×100 ml) and dried over anhydrous MgSO₄. The dry organic phase was concentrated at reduced pressure and two entrainments were carried out on the resulting concentrate at reduced pressure with 50 ml of heptane at each entrainment, thereby providing 4.83 g of (R)-2-chloro-N-[2-(4-methoxyphenyl)-1-methylethyl] acetamide as a whitish solid.

Yield: 66.0% (R/S)=60/40

### EXAMPLE 4

### Preparation of (R)-2-chloro-N-[2-(4-methoxyphenyl)-1-methylethyl]acetamide with Novozym 435 used Previously (Enzyme/Substrate Ratio=10% w/w).

1.00 g of recovered Novozym 435 and 100 ml of TBME were placed in a 500 ml flask under N₂ atmosphere. A solution of 32.40 ml (37.07 g; 0.3025 mol; 5 eq) of ethyl chloroacetate in 100 of TBME, and finally a solution of 10.00 g (0.0605 mol; 1 eq) of (R,S)-4-methoxyamphetamine in 100 ml of TBME, were added. The mixture was stirred at room temperature for 5 hours, following which it was filtered, and the retained solid, i.e. a mixture of Novozym 435 and 4-methoxy-α-methylbenzene ethanammonium chloroacetate, was washed with 100 ml of TBME. The filtered solution was washed with 50 ml of H₂SO₄ 0.5 M and dried over anhydrous MgSO₄. The dry organic phase was concentrated at reduced pressure and two entrainments were carried out on the resulting concentrate at reduced pressure with 100 ml of heptane at each entrainment, thereby providing 5.59 g of (R)-2-chloro-N-[2-(4-methoxyphenyl)-1-methylethyl]acetamide as a whitish solid.

Yield: 38.3% (R/S)=96.1/3.9

### EXAMPLE 5

### Preparation of Enantiomerically Pure (R)-2-chloro-N-[2-(4-methoxyphenyl)-1-methylethyl]acetamide with Novozym 435 Previously used in a Single Operation (Enzyme/Substrate Ratio=10% w/w)

Onto a mixture of 1.92 kg of Novozym 435 (moist products of TBME, estimated at dryness: 0.80 kg), 160 lt of TBME and 29.70 kg (242.35 mol; 5 eq) of ethyl chloroacetate, under nitrogen atmosphere, was added 67.20 kg of a solution of (R,S)-4-methoxyamphetamine in TBME (11.9%; w/w; estimated at dryness: 8.00 kg; 48.43 mol; 1 eq) maintaining T=15/30° C. The resulting suspension was stirred for 6 hours, following which it was filtered through nutcha and the retained solid, i.e. a mixture of Novozym 435 and 4-methoxy-α-methylbenzene ethanammonium chloroacetate, was washed with 80 lt of TBME. The liquids of the filtrate were washed with a mixture of 2.24 lt of H₂SO₄ (98%) and 78 lt of H₂₀. The resulting organic phase was concentrated to dryness at reduced pressure, for which entrainments with heptane (6×30 lt) were carried out. Onto the obtained concentration residue, 65 lt of TBME and 0.40 kg of Hyflo supercell were added, and this was heated at reflux (T=50/55° C.) for 15 minutes, following which it was filtered and the retained solid was washed with 4.40 lt of TBME. The filtered solution was cooled to T=0/10° C., carrying out seedings with (R)-2-chloro-N-[2-(4-methoxyphenyl)-1-methylethyl] acetamide (R/S ratio=99.47/0.53) during the cooling, and it was maintained within that range for 30 minutes. The resulting white suspension was then filtered through nutcha and the retained solid was washed with 2.96 lt of cold TBME. The moist product obtained was dried at T=40° C. in vacuo to constant weight, thereby providing 2.54 kg of dry product.

Yield: 21.7% (R/S)=99.64/0.36

### EXAMPLE 6

### Preparation of (R)-2-chloro-N-[2-(4-methoxyphenyl)-1-methylethyl]acetamide with Lipozime RM IM (Enzyme/Substrate Ratio=10% w/w).

0.50 g of Lipozime RM IM and 50 ml of TBME were placed in a 250 ml flask under N₂ atmosphere. A solution of 16.20 ml (18.57 g; 0.1515 mol; 5 eq) of ethyl chloroacetate in 50 ml of TBME were then added, followed by a solution of 5.00 g (0.0303 mol; 1 eq) of (R,S)-4-methoxyamphetamine in 50 ml of TBME. The reaction mixture was stirred at T=20-25° C. for 64 h, following which it was filtered and the retained solid, i.e. a mixture of Novozym 435 and 4-methoxy-α-methylbenzene ethanammonium chloroacetate, was washed with 50 ml of TBME. The filtered solution was washed with 25 ml of H₂SO₄ 0.5 M and dried over anhydrous MgSO₄. The dry organic phase was concentrated at reduced pressure and two entrainments were carried out on the resulting concentrate at reduced pressure with 100 ml of heptane at each entrainment, thereby providing 0.61 g of (R)-2-chloro-N-[2-(4-methoxyphenyl)-1-methylethyl]acetamide as yellow crystals of oily appearance.

Yield: 8.5% (R/S)=91.6/8.4

### EXAMPLE 7

### Preparation of (R)-2-chloro-N-[2-(4-methoxyphenyl)-1-methylethyl]acetamide with Lipozime TL IM (Enzyme/Substrate Ratio=10% w/w)

0.50 g of Lipozime RM IM and 50 ml of TBME were placed in a 250 ml flask under N₂ atmosphere. A solution of 16.20 ml (18.57 g; 0.1515 mol; 5 eq) of ethyl chloroacetate in 50 ml of TBME was then added, followed by a solution of 5.00 g (0.0303 mol; 1 eq) of (R,S)-4-methoxyamphetamine in 50 ml of TBME. The reaction mixture was stirred at T=20-25° C. for 41 h, following which it was filtered and the retained solid, which is a mixture of Novozym 435 and 4-methoxy-α-methylbenzene ethanammonium chloroacetate, was washed with 50 ml of TBME. The filtered solution was washed with 25 ml of H₂SO₄ 0.5 M and dried over anhydrous MgSO₄. The dry organic phase was concentrated at reduced pressure and two entrainments were carried out on the resulting concentrate at reduced pressure with 100 ml of heptane at each entrainment, thereby providing 2.17 g of (R)-2-chloro-N-[2-(4-methoxyphenyl)-1-methylethyl]acetamide as yellow crystals of oily appearance.

Yield: 29.6% (R/S)=92/8

### EXAMPLE 8

### Purification of (R)-2-chloro-N-[2-(4-methoxyphenyl)-1-methylethyl]acetamide in TBME

200 g of (R)-2-chloro-N-[2-(4-methoxyphenyl)-1-methylethyl]acetamide (R/S ratio=91.3/8.7) and 2.40 lt of TBME were placed in a 3 lt flask with coupled coolant. The mixture was heated at reflux (T=50/55° C.) until the product had dissolved, following which it was hot-filtered through a prelayer of Hyflo supercell, the prelayer was washed with 100 ml of TBME and the filtered solution seeded with (R)-2-chloro-N-[2-(4-methoxyphenyl)-1-methylethyl]acetamide (R/S ratio=98.7/1.3) for the cooling. The resulting suspension was maintained at T=0/10° C. for 30 minutes, then filtered and washed with cold TBME (2×50 ml). The solid obtained was dried at T=40° C. in vacuo, thereby providing 106.50 g of dry product.

Yield: 53.2% (R/S)=96.5/3.5

18.76 g of the product thus obtained was recrystallised in 300 ml of TBME, seeding with (R)-2-chloro-N-[2-(4-methoxyphenyl)-1-methylethyl]acetamide (R/S ratio=98.7/1.3) during the cooling. The suspension was maintained at T=0/10° C. for 30 minutes, following which it was filtered and washed with 4 ml of cold TBME. The resulting solid was dried at T=40° C. in vacuo, thereby providing 7, 83 g of dry product.

Yield: 41.7% (R/S)=99.77/0.23

### EXAMPLE 9

### Preparation of Tamsulosin Hydrochloride

### a) Preparation of (R)-N-[2-(3-aminosulphonyl-4-methoxyphenyl)-1-methylethyl]-2-chloro-acetamide from (R)-2-chloro-N-[2-(4-methoxyphenyl)-1-methylethyl]acetamide

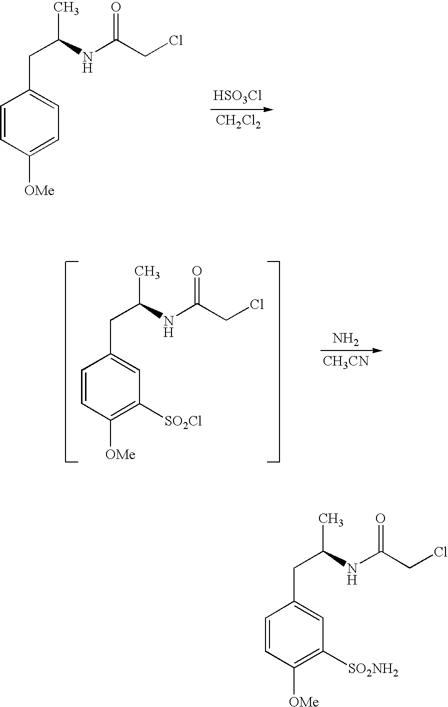

A solution of 2.50 kg (10.35 mol; 1 eq) of (R)-2-chloro-N-[2-(4-methoxyphenyl)-1-methylethyl]acetamide in 25 lt of CH₂Cl₂ was added under nitrogen atmosphere to 6.03 kg (51.75 mol; 5 eq) of chlorosulphonic acid, previously cooled to T=−5/5° C. The addition was carried out while maintaining the temperature within the range −5/5° C. After completing the addition, the mixture was heated to T=15/25° C. and was maintained under those conditions for 1 hour. Once that period had elapsed, it was cooled to T=−10/0° C. and added to a mixture of 75 lt of H₂O and 38.8 lt of CH₂Cl₂, maintaining the temperature of the mixture within the range T=25/35° C. The organic phase was then decanted and concentrated at reduced pressure until a thick paste was obtained. 26 lt of acetonitrile was added to the distillation residue and this was stirred to homogenise the mixture, to provide 22.58 kg of solution with a content by weight of (R)-N-[2-(3-chlorosulphonyl-4-methoxyphenyl)-1-methylethyl]-2-chloro-acetamide of 11.85% (2.67 kg).

The solution of (R)-N-[2-(3-chlorosulphonyl-4-methoxyphenyl)-1-methylethyl]-2-chloro-acetamide was cooled to T=−10/0° C. and 1.74 lt of NH₃ (25%) was added, maintaining the temperature within the range stated. After completing the addition, the reaction was maintained at T=−10/0° C. for 1 hour and 40.5 lt of H₂O was then added, maintaining T<10° C. The pH of the mixture was then adjusted between 5.0 and 6.0 with 376 ml of HCl (35%), concentrated at reduced pressure in order to eliminate the CH₃CN and the resulting suspension was cooled to T=10/20° C. for 2 hours, following which it was filtered and washed with 5 lt of H₂O, providing a white solid which was dried at 50° C. in vacuo to K.F.<0.2%.

Weight=2.26 kg

Yield=68.1%

### b) Preparation of (R)-N-[2-(3-aminosulphonyl-4-methoxyphenyl)-1-methylethyl]-2-(2-ethoxyphenoxy)-acetamide from (R)-N-[2-(3-aminosulphonyl-4-methoxyphenyl)-1-methylethyl]-2-chloroacetamide

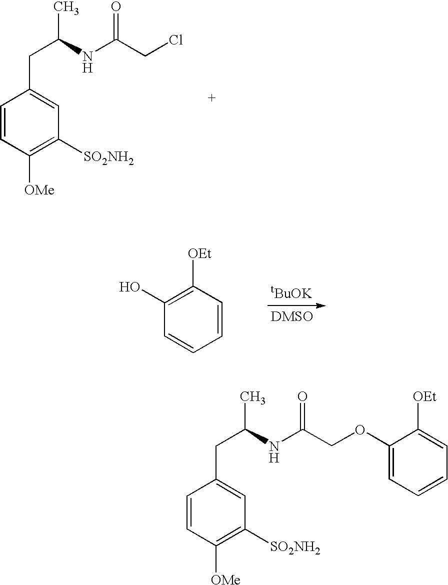

1.73 kg (12.52 mol; 2.5 eq) of 2-ethoxyphenol was added to a suspension of 2.24 kg (19.96 mol; 4 eq) of t-BuOK in 6.72 lt of DMSO, under nitrogen atmosphere, maintaining T=20/30° C. 1.60 kg (4.99 mol; 1 eq) of (R)-N-[2-(3-aminosulphonyl-4-methoxyphenyl)-1-methylethyl]-2-chloro-acetamide were then added, maintaining T=20/30° C. After completing the addition, the mixture was maintained at T=25/35° C. for 2 hours, upon which it was poured while maintaining T=25/35° C. onto a mixture of 13.1 lt of H₂O and 6.56 lt of heptane. The pH was then adjusted to 1.0-2.0 with 1.39 it of HCl (35%) and the oil in suspension obtained was heated to T=50/60° C. for 1 hour to break it down. Finally, the resulting suspension was cooled to T=0/10° C. for 1 hour, filtered and washed with a mixture of 2.24 it of H₂O and 1.15 it of heptane.

The moist compound (R)-N-[2-(3-aminosulphonyl-4-methoxyphenyl)-1-methylethyl]-2-(2-ethoxyphenoxy)-acetamide thus obtained was suspended over 11.2 it of heptane and was heated at reflux (T=95-100° C.) for 1 hour, upon which it was cooled to T=0/10° C. and thus maintained for 1 hour. Finally, the suspension was filtered, the solid was washed with 3.20 it of heptane and dried at 60° C. in vacuo.

Weight=1.84 kg

Yield=87.2%

### c) Preparation of tamsulosin hydrochloride from (R)-N-[2-(3-aminosulphonyl-4-methoxyphenyl)-1-methylethyl]-2-(2-ethoxyphenoxy)-acetamide

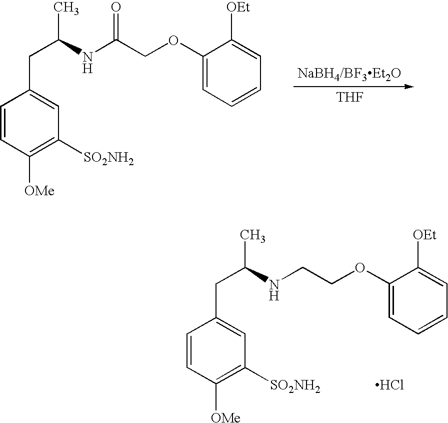

2.74 kg (19.31 mol; 5.1 eq) of boron etherate trifluoride was added to a suspension of 0.573 kg (15.15 mol; 4 eq) of sodium borohydride in 8 it of THF, under nitrogen atmosphere, maintaining T=10/20° C. A solution of 1.60 kg (3.79 mol; 1 eq) of (R)-N-[2-(3-aminosulphonyl-4-methoxyphenyl)-1-methylethyl]-2-(2-ethoxyphenoxy)-acetamide in 8 lt of THF was then added, maintaining T=10/20° C., and was heated at reflux (T=55/60° C.) for 2 hours. 3.20 lt of H₂O was then added slowly, maintaining T=20/40° C., followed by 1.45 lt of HCl (35%). The resulting mixture was heated at reflux (T=55/65° C.) for 30 minutes and then left to cool to T=20/40° C. and concentrated at reduced pressure to eliminate the THF. 24 lt of H₂O was then added and it was concentrated again to a final volume of 30 lt. 8 lt of CH₂Cl₂ was added to the concentrate obtained and this was adjusted to pH=8.0-9.0 with 3.3 lt of NaOH (30%). The mixture was heated to T=30/40° C. and 24 lt of water were added. The organic phase was decanted, and a further 8 lt of CH₂Cl₂ was added to the aqueous phase. The combined organic extracts were washed with 10 lt of H₂O and concentrated at reduced pressure to a final volume of 2 lt. 14.4 lt of acetone was added to the concentrate obtained and was heated to T=40/50° C. The mixture was adjusted to pH=1.0-2.0 with 0.32 lt of HCl (35%) and the resulting suspension cooled at T=−5/5° C. for 1 hour, upon which it was filtered and washed with 1.6 lt of acetone. Finally, the moist solid was dried at T=40° C. in vacuo.

Weight=1.30 kg

Yield=77%

1. Process for the preparation of (R)-2-halo-N-[2-(4-methoxyphenyl)-1-methylethyl]acetamide, of formula III, wherein X is Cl, Br, I: 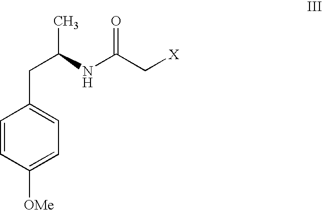
wherein it comprises the enantioselective acylation reaction of the (R,S)-4-methoxyamphetamine of formula: 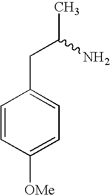
with an acylating agent in the presence of an enzyme.

2. Process according to claim 1, wherein the enzyme is a lipase.

3. Process according to claim 1, wherein the enzyme is the lipase Novozym 435.

4. Process according to claim 1, wherein the enzyme is the lipase Lipozime RM IM.

5. Process according to claim 1, wherein the enzyme is the lipase Lipozime TL IM.

6. Process according to claim 1, wherein the acylating agent is a haloalklyl ester.

7. Process according to claim 6, wherein the acylating agent is ethyl chloroacetate.

8. Process according to claim 1, wherein the weight/weight ratio between enzyme and substrate is 5-20%.

9. Process according to claim 8, wherein the weight/weight ratio between enzyme and substrate is 10-16%.

10. Process according to claims claim 8, wherein the weight/weight ratio between enzyme and substrate is 10%.

11. Process according to claim 1, which further includes the reaction of compound III with chlorosulphonic acid and ammonia to give a compound of formula IV, 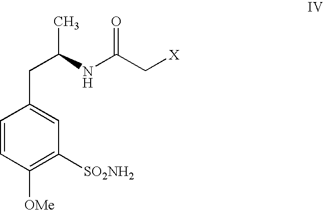
which is reacted with 2-ethoxyphenol in the presence of a strong base and an organic solvent to provide compound V, 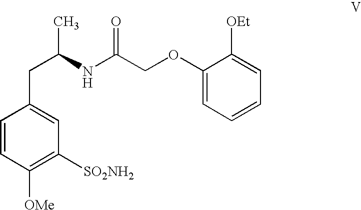
the compound V obtained is reduced with a reducing agent, and the subsequent treatment with hydrochloric acid yields tamsulosin hydrochloride.

12. Process according to claim 1, wherein the reaction is carried out in an inert organic solvent.

13. Process according to claim 12, wherein the inert organic solvent is one of the ether type.

14. Process according to claim 13, wherein the organic solvent is t-butyl methyl ether.

15. Process according to claim 1, wherein the reaction is carried out at a temperature between 0-70° C.

16. Process according to claim 15, wherein the reaction is carried out at a temperature between 0-5° C. or 15-30° C.

17. Process for preparing tamsulosin hydrochloride comprising: (R)-2-chloro-N-[2-(4-methoxyphenyl)-1-methylethyl] acetamide, of formula III: [chemical structure]
